(19) United States
(12) Patent Application Publication
(10) Pub. No.: US 2025/0032320 A1
(43) Pub. Date: Jan. 30, 2025

(54) WELDING SHIELD

(71) Applicant: TECHNOLOGICAL UNIVERSITY DUBLIN, Dublin (IE)

(72) Inventors: Niall MURPHY, Dublin (IE); Stephen COX, Dublin (IE)

(21) Appl. No.: 18/708,785

(22) PCT Filed: Nov. 9, 2022
(86) PCT No.: PCT/EP2022/081369

§ 371 Date: May 9, 2024

(30) Foreign Application Priority Data
Nov. 9, 2021 (GB) 2116062.7

(51) Int. Cl. A61F 9/06

(52) U.S. Cl. CPC A61F 9/068

(57) ABSTRACT

Disclosed is a welding shield that includes an air supply conduit for receiving and supplying ambient air into an internal portion of the welding shield, a sensor provided within the air supply conduit at an inlet of the conduit for detecting presence of one or more harmful gases in the ambient air, before the ambient air reaches the wearer for inhaling, and an alerting system operably coupled to the sensor for alerting the wearer upon detection of the one or more harmful gases in the ambient air.

### FIELD OF INVENTION

The present invention relates to a welding shield, and more specifically to detecting harmful gases in the ambient air inside the welding shield.

### BACKGROUND

Welding shields are typically used in mechanical and industrial industries to protect welders from harmful irradiation emitted from the welding arc and from splashes, sparks and particles that may be ejected from a welding area. Welding shields may be suspended on the head of a wearer, so that the wearer has both hands available for welding and handling of workpieces.

However, sometimes harmful fumes and gases may enter the welding shield through a breathing passageway of the welding shield without the user knowing about them. The wearer can continue inhaling the harmful gases inside the welding shield as not all fumes and gases can be seen or tasted while welding. Some of the fumes and gases inhaled by the welder can cause serious health problems or can in some cases be fatal. Examples of such gases include, but are not limited to argon, carbon dioxide, carbon monoxide, helium, iron oxide, nitrogen, aluminium, chromium, lead, manganese, and mild steel. Examples of diseases that can be caused due to inhaling of such gases include lung disease, memory loss, fatigue, bronchitis, Parkinson's disease, cancer, brain, and nervous system malfunctioning. Also, some of the consumables used in the welding process may cause sickness and cancer. Examples of such consumables include tungsten, welding MIG wire, and arc rods.

US 2017/136268 discloses an air-feed welding shield that provides an air quality monitor in said helmet whereby a parameter indicative of air quality is displayed in, on or adjacent said vision panel. The air-feed welding shield provides an indication of air quality of the air generated by the in-built respirator. Said air-feed welding shield is limited to detecting low air quality and does not disclose detecting not specific toxins in the inhaled air.

WO 2015/036652 also discloses an air-feed mask that includes a carbon-dioxide sensor. GB 2565120 refers to a clip-on sensor that can be clipped onto the welder's clothing, torch, or helmet. CN 109820272 discloses a multifunctional safety helmet for construction and pharma, and does not specify a welding helmet. WO 2020/104815 discloses an oxygen sensor that is housed in the powered air personal respirator and not within the welding shield.

Each of the above-mentioned documents disclose an air-feed mask which includes an air respirator. None of above-mentioned prior art disclose detecting harmful gases in the ambient air inside a conventional welding shield, and alerting the user immediately.

In view of the above, there is a need for a system that may sense the presence of harmful and dangerous gases inside the welding shield, and may alert the wearer, so that the wearer may take a corrective action immediately.

### SUMMARY

According to the invention there is provided, as set out in the appended claims, a welding shield as defined in any of Claims 1 to 15.

In one embodiment there is provided a welding shield comprising a source of air flow inside the welding shield; a sensor for detecting presence of one or more harmful gases that have entered inside the welding shield through the source; and an alerting system for alerting a wearer user upon detection of the one or more harmful gases inside the welding shield.

According to the invention, there is further provided a welding shield that includes an air supply conduit for receiving and supplying ambient air into an internal portion of the welding shield; a sensor provided within the air supply conduit at an inlet of the conduit for detecting presence of one or more harmful gases in the ambient air, before the ambient air reaches the wearer for inhaling; and an alerting system operably coupled to the sensor for alerting the wearer upon detection of the one or more harmful gases in the ambient air.

In an embodiment of the present invention, the welding shield further includes a fan provided adjacent to the air supply conduit and the sensor for drawing in the ambient air into the internal portion of the welding shield.

In an embodiment of the present invention, the air supply conduit is one of: a dedicated vent or an air passageway.

In an embodiment of the present invention, the welding shield comprises a fan to maintain an air flow inside the welding shield, wherein the fan is provided at a top internal portion of the welding shield to circulate air around to ensure that the one or more harmful gases move around in the welding shield, and wherein the fan prevents the sensor from generating a false alert due to wearer's exhaled breath.

In an embodiment of the present invention wherein the sensor is configured to monitor the level of oxygen in the air. Suitably when the oxygen level drops below a certain level an alert is generated to the user.

In an embodiment of the present invention the sensor is attached to an internal portion of the welding shield, at a position above eyes of the wearer.

In an embodiment of the present invention, the source is an air passageway for enabling the wearer to be able to breath while wearing the welding shield.

In an embodiment of the present invention, the source is a gap between an outer inner surface of the welding shield and a facial portion of the wearer.

In an embodiment of the present invention, the sensor is placed in a supply pipe of the source. It will be appreciated that the sensor can be remote from the welding shield in a backpack source feeding air directly to the helmet.

In an embodiment of the present invention, the sensor is configured to detect at least one of: Carbon Monoxide (CO), Carbon dioxide (CO₂), Manganese, Hydrogen Fluoride, Nitrogen Oxides, Ozone, Oxygen, Nickel, and zinc inside the welding shield.

In an embodiment of the present invention, the alerting system includes a flashing light for alerting the wearer upon detection of the one or more harmful gases inside the welding shield.

In an embodiment of the present invention, the alerting system includes an alarm for alerting the wearer upon detection of the one or more harmful gases inside the welding shield. Suitably the alarm can be a visual light, an audio alarm or a tactile vibrating alert.

In an embodiment of the present invention, the sensor is powered using at least one of: lithium batteries and solar power.

In an embodiment of the present invention, the alerting system further includes a glass screen for enabling the wearer to see through the welding shield while performing welding operation.

In an embodiment of the present invention, the glass screen includes an auto darkening lens, that is configured to automatically darken itself to protect the wearer from becoming blinded by arc ray during welding.

In one embodiment of the present invention, the welding shield comprises a transmitter/receiver and configured to transmit an alert to a central controller, in the event an alert is triggered by the sensor.

In one embodiment of the present invention, the fan is provided at a top internal portion of the welding shield to circulate air around to ensure the fan prevents misting of the inner surface of the protective auto darkening lens due to wearer's exhaled breath. The fan can be configured to prevent fogging or misting of the lens to enable the wearer to see more clearly.

The welding shield of the present invention comprises a fixed sensor in front of a small fan in a chamber at its entry point. The sensor may be placed within the air supply conduit at an inlet of the air supply conduit. The welding shield of the present invention ideally provides a position to place the sensor inside the welding shield to detect the harmful gases in the ambient air before the ambient air reaches the wearer for inhaling. The sensor and fan are provided in the same compartment, so that the fan draws in the ambient air towards the sensor. The welding shield of the present invention is a conventional welding shield and not an air-feed welding shield. The welding shield of present invention solves the problem of harmful gases inside the welding shield, by providing a welding shield that that may sense/detect the presence of harmful and dangerous gases in the ambient air, so that the wearer may take a corrective action immediately.

### BRIEF DESCRIPTION OF THE DRAWINGS

The invention will be more clearly understood from the following description of an embodiment thereof, given by way of example only, with reference to the accompanying drawings, in which:

FIG. 1A illustrates a side view of a welding shield, in accordance with an embodiment of the present invention;

FIG. 1B illustrates a front view of the welding shield of FIG. 1, in accordance with an embodiment of the present invention;

FIG. 2A illustrates a side view of another welding shield, in accordance with an embodiment of the present invention;

FIG. 2B illustrates a front view of the welding shield of FIG. 2A, in accordance with an embodiment of the present invention;

FIG. 3A illustrates a perspective view of a welding shield, in accordance with another embodiment of the present invention;

FIG. 3B illustrates an internal view of the welding shield of FIG. 2A, in accordance with another embodiment of the present invention; and

FIG. 4 illustrates a perspective view of a welding shield, in accordance with yet another embodiment of the present invention.

### DETAILED DESCRIPTION OF DRAWINGS

The invention is not limited to the embodiments hereinbefore described but may be varied in both construction and detail.

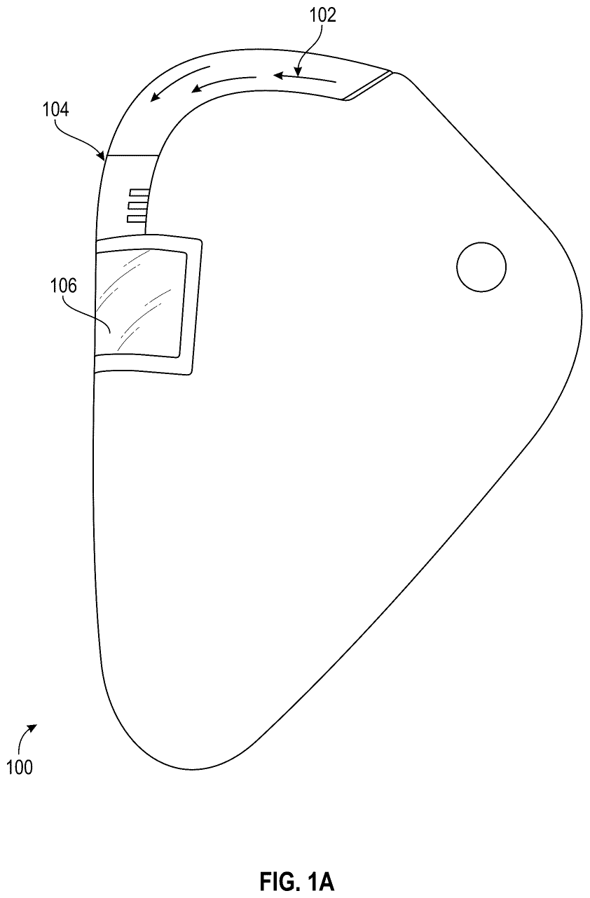

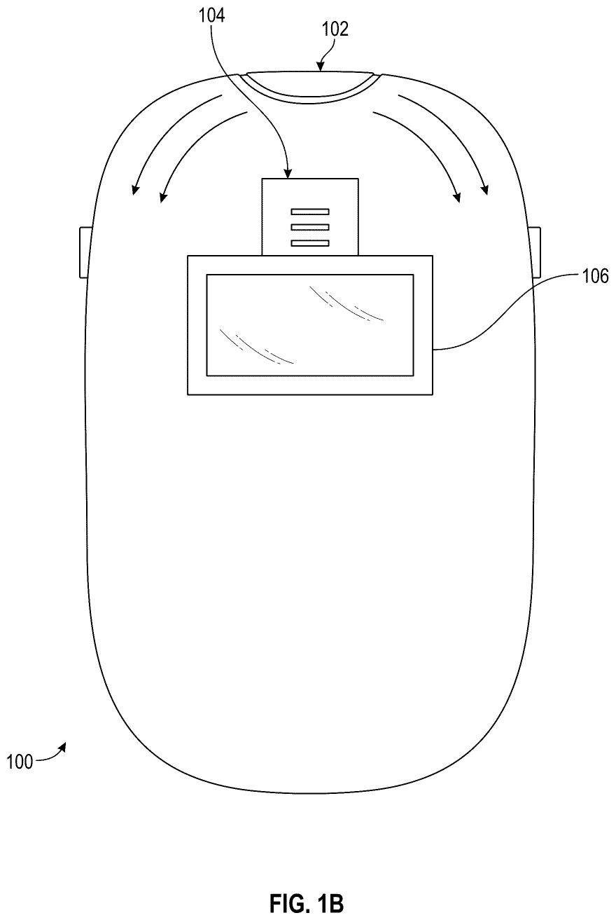

Referring to FIGS. 1A and 1B, a welding shield 100 is disclosed. The welding shield 100 is in the form of a helmet that can be worn by a user or wearer while performing welding operation. The welding shield 100 includes a source 102 of air flow, a detector 104 for detecting presence of harmful gases inside the welding shield 100, and a glass screen 106 for enabling a user to see through the welding shield 100 for performing welding operation.

The source 102 may be an air passageway for enabling a wearer to be able to breath while wearing the welding shield 100. Alternatively, the source 102 may be a gap between an outer surface of the welding shield 100 and a face of the user. While the user breathes the oxygen through the source 102, there is a possibility of the user inhaling harmful gases during the welding process.

Although, in the preferred embodiment, the source 102 may be provided at a top portion of the welding shield 100, it may be apparent to one of ordinary skill in the art, that the source 102 may be provided at any another portion of the welding shield 100.

The detector 104 may be small device attached to an internal portion of the welding shield 100, and may be located near to the chin area, close to the mouth and nose. Preferably, the detector 104 is placed at a position above the eyes of a user, as positioning the detector 104 near to the mouth area can lead to generation of a false alarm due to exhaled breath. The alarm can also cause the sensor or part, or the whole, of the helmet to vibrate to inform the user of a potential danger.

In the context of the present invention, the detector 104 includes a sensor that is configured to detect the harmful substances generated from various types of welding processes. The sensor is configured to detect a wide range of fumes and gases inside the welding shield. The sensor is configured to detect high/low levels of for example, Carbon Monoxide (CO), Carbon dioxide (CO₂), Manganese, Hydrogen Fluoride, Nitrogen Oxides, Ozone, Oxygen, Nickel, and zinc. The sensor can also be configured to detect the level of oxygen. If the level is below or above a desired level an alert is generated to the user.

The detector 104 may further include an alerting system to alert to the user regarding the presence or absence of these hazardous gases and fumes. In one example, the alerting system may include a flashing light to alert the wearer regarding the presence of the fumes and gases inside the welding head shield 100. In another example, the alerting system may include an alarm that may make a sound of about 90 dBs, and preferably in the range of 50 dB to 100 dB, to alert the welder of the fumes and gases that have entered the welding shield 100. The alerting system provides the user an opportunity to either move his/her position, stop welding until the fumes and gases have dissipated or use an alternative system to remove the toxins. The detector 104 may be housed in a casing that may not interfere with the comfort or the vision of the welder and it may be easily accessible for a battery change if needed.

In an embodiment of the present invention, the glass screen 106 includes an auto darkening lens, that is configured to automatically darken itself to protect the welder from becoming blinded by the arc ray during welding. The auto darkening lens automatically darkens when a weld is struck to protect the welder's eyes from these rays.

In an embodiment of the present invention, the auto darkening lens is powered using solar power through a number of light sensors. In entirety, the detector 104 is powered using a combination of lithium batteries and a combination of solar power, which would also be good as the power needed would be increased with the fume detector, and this combination would extend the life of the welding shield 100.

Although not shown, the welding shield 100 comprises a small fan attached to an internal portion of the welding shield 100, to keep the air flowing in the shield 100. The speed of air flow from the fan is set such that not to cause a discomfort to the wearer of the head shield 100. The fan aids the sensor from sending a false alarm because of the user's exhaled breath. The fan may be powered from a battery and may also be powered from the cells that power the auto darkening solar powered screen 106. Preferably, the fan is provided at a top of the welding shield 100 to circulate air around to ensure that the toxic gases and fumes are moving and not lying in the welding shield 100. The fan is configured to draw ambient air over the sensor. Thus, the presence of the fan reduces the possibility of false alarms.

It will be appreciated the positioning the detector/sensor can be placed in the supply pipe of an external air source, or in the supply unit of an air source, along with the current position within the helmet 100.

In another embodiment the helmet can be configured with a transmitter/receiver. In the event an alert is triggered by the sensor the alert can be transmitted to a central controller informing a third party that an alert is generated. This can be important in the case the user is incapacitated and a third party can be sent to make sure the user is safe. In another embodiment the welding shield can also be equipped with a camera device that can transmit the information to the central controller.

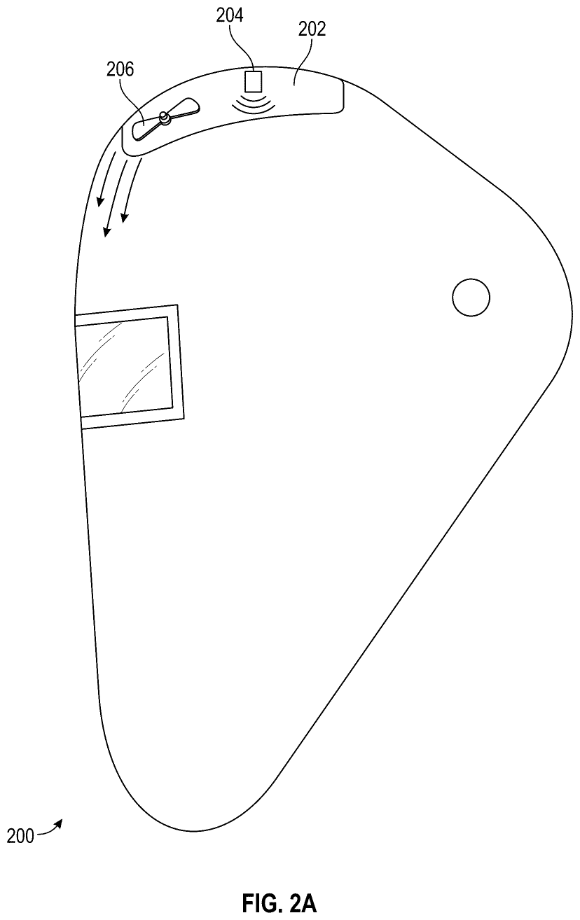

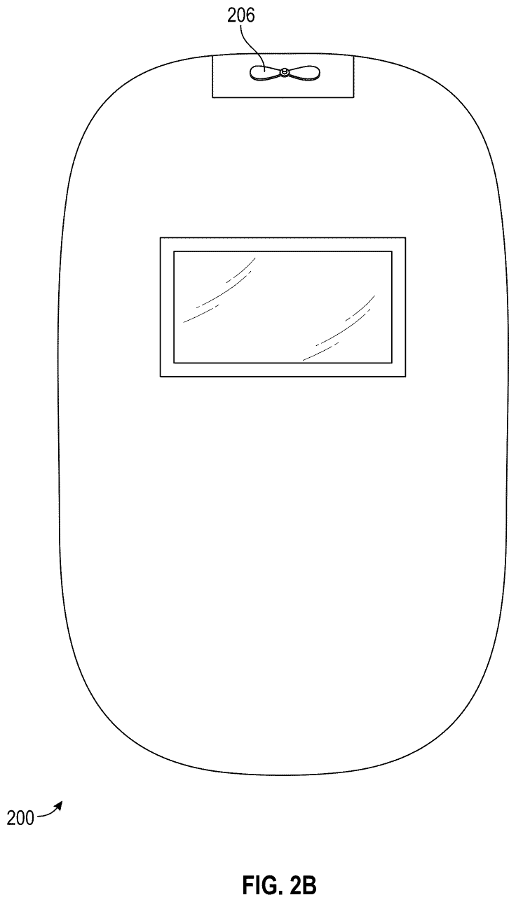

FIG. 2A illustrates a side view of another welding shield 200, in accordance with an embodiment of the present invention. FIG. 2B illustrates a front view of the welding shield of FIG. 2A, in accordance with an embodiment of the present invention.

The welding shield 200 includes an air supply conduit 202 for enabling a wearer of the welding shield to inhale ambient air. Preferably, the air supply conduit is provided at a top internal portion of the welding shield, and is one of a dedicated vent or an air passageway.

There is provided a sensor 204 within the air supply conduit 202 at an inlet of the conduit 202 or detecting presence of one or more harmful gases in the ambient air, before the ambient air reaches the wearer for inhaling. Preferably, the sensor 204 is attached to an internal portion of the welding shield, at a position above eyes of the wearer. The sensor 204 may be similar to the afore-mentioned detector 104 (a shown in FIG. 1).

There is further provided a fan 206 adjacent to the sensor 204 for drawing in the ambient air into the internal portion of the welding shield. Essentially, the fan 206 facilitates directing the ambient air towards the sensor 204.

The fan 206 further provides air circulation to enable movement of the one or more harmful gases in the welding shield 204, and prevent the sensor 204 from generating a false alert due to wearer's exhaled breath. The fan 206 keeps the wearer's exhaled breath away from the sensor 204. The sensor 204 may include an alerting system for alerting the wearer upon detection of the one or more harmful gases in the inhaled air.

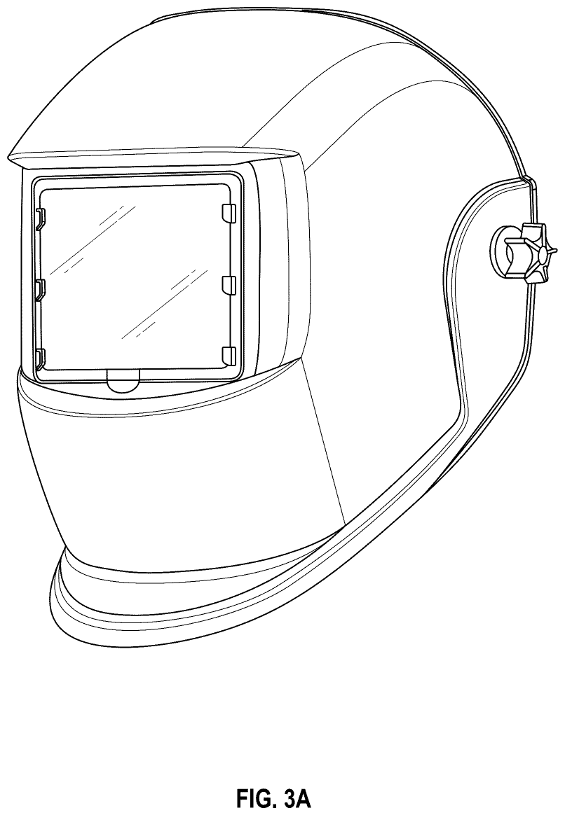

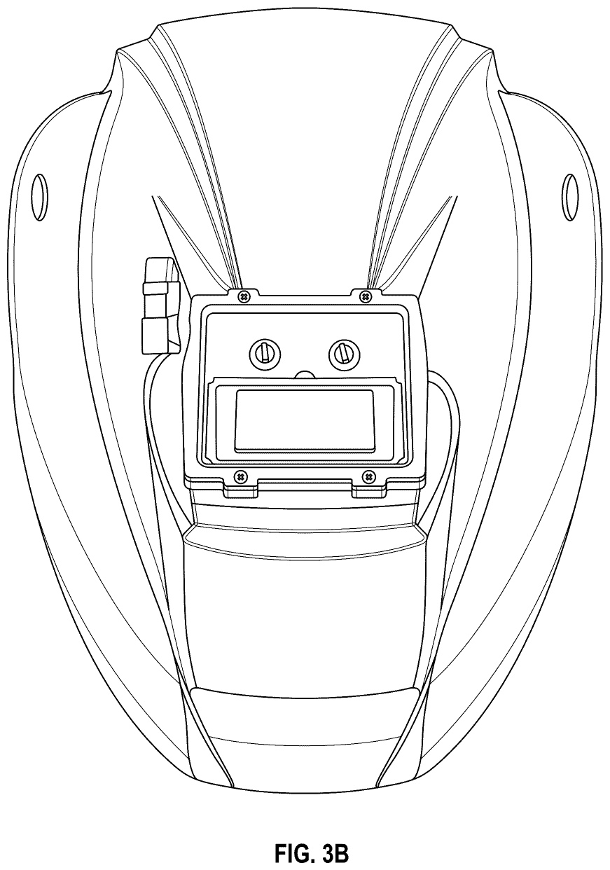

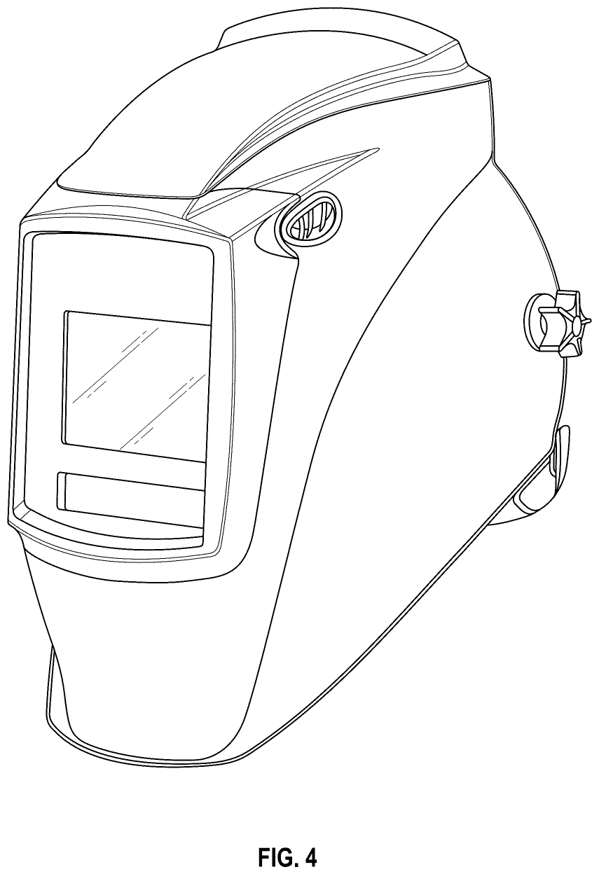

It is to be noted that the welding shields 100 and 200 disclosed herein are conventional welding shields, and not air-feed welding shields, and does not include a separate respirator for enabling a user to breathe. Rather, the welding shields 100 and 200 detect harmful gases in the ambient air before the ambient air reaches the wearer for inhaling. The designs of the welding shield 100 as disclosed in FIGS. 1A, 1B, 2A and 2B form a basis for manufacturing the various welding shields as disclosed in FIGS. 3A, 3B and 4.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g., a memory stick or hard disk. The carrier may be an electrical or optical signal which may be transmitted via an electrical or an optical cable or by radio or other means.

In the specification the terms “comprise, comprises, comprised and comprising” or any variation thereof and the terms include, includes, included and including” or any variation thereof are considered to be totally interchangeable, and they should all be afforded the widest possible interpretation and vice versa.

The invention is not limited to the embodiments hereinbefore described but may be varied in both construction and detail.

1. A welding shield comprising:
an air supply conduit for receiving and supplying ambient air into an internal portion of the welding shield;
a sensor provided within the air supply conduit at an inlet of the conduit for detecting presence of one or more harmful gases in the ambient air, before the ambient air reaches the wearer for inhaling; and
an alerting system operably coupled to the sensor for alerting the wearer upon detection of the one or more harmful gases in the ambient air.

2. The welding shield of claim 1 further comprising a fan provided adjacent to the air supply conduit and the sensor for drawing in the ambient air into the internal portion of the welding shield.

3. The welding shield of claim 1, wherein the fan is provided at a top internal portion of the welding shield to circulate air around to ensure that the one or more harmful gases move around in the welding shield, and wherein the fan prevents the sensor from generating a false alert due to wearer's exhaled breath.

4. The welding shield of claim 1, wherein the sensor is attached to an internal portion of the welding shield, at a position above eyes of the wearer.

5. The welding shield of claim 1, wherein the air supply conduit is one of: a dedicated vent or an air passageway.

6. The welding shield of claim 1, wherein the sensor is configured to detect at least one of: Carbon Monoxide (CO), Carbon dioxide (CO2), Manganese, Hydrogen Fluoride, Nitrogen Oxides, Ozone, Oxygen, Nickel, and zinc inside the welding shield.

7. The welding shield of claim 1, wherein the alerting system includes a flashing light for alerting the wearer upon detection of the one or more harmful gases inside the welding shield.

8. The welding shield of claim 1, wherein the alerting system includes an alarm for alerting the wearer upon detection of the one or more harmful gases inside the welding shield.

9. The welding shield of claim 1, wherein the sensor is powered using at least one of: lithium batteries and solar power.

10. The welding shield of claim 1 further comprising a glass screen for enabling the wearer to see through the welding shield while performing welding operation.

11. The welding shield as claimed in claim 10, wherein the glass screen includes an auto darkening lens, that is configured to automatically darken itself to protect the wearer from becoming blinded by arc ray during welding.

12. The welding shield of claim 1, wherein the sensor is configured to monitor the level of oxygen in air and when the oxygen level drops below a certain level an alert is generated.

13. The welding shield of claim 1, wherein the sensor is remotely located from the welding shield in a backpack source feeding air directly to the welding shield.

14. The welding shield of claim 1 further comprising a transmitter/receiver configured to transmit an alert to a central controller, in the event an alert is triggered by the sensor.

15. The welding shield of claim 1, wherein the fan is provided at a top internal portion of the welding shield to circulate air around to ensure the fan prevents misting of the inner surface of the protective auto darkening lens due to wearer's exhaled breath.
